(19) United States
(12) Patent Application Publication
(10) Pub. No.: US 2015/0045883 A1
(43) Pub. Date: Feb. 12, 2015

(54) Middle Ear Prosthesis with Recesses for Applicator Engagement

(71) Applicant: Mark Matthew Scheurer, Coronado, CA (US)

(72) Inventor: Mark Matthew Scheurer, Coronado, CA (US)

(21) Appl. No.: 13/959,832

(22) Filed: Aug. 6, 2013

(51) Int. Cl. A61F 2/18

(52) U.S. Cl. CPC A61F 2/18
USPC 623/10

(57) ABSTRACT

A middle ear prosthesis comprises an ossicular attachment component contiguous with a vibration transmission element, the attachment component comprising a loop or other structure to surround and attach the prosthesis to an ossicle. The prosthesis has a plurality of recesses designed to be securely engaged by the jaws of an applicator or forceps into the recesses. This engagement can allow the attachment element to be opened or closed via an expanding or compressing force from the applicator. The recesses may be squared off in appearance or rounded and may have a complex shape to more precisely accommodate the jaws of the applicator. The applicator can also be angled, configured, and dimensioned to provide an optimum engagement with the prosthesis.

### CROSS-REFERENCE TO RELATED APPLICATIONS

This application claims priority of the following provisional applications: Provisional Application Ser. No. 61/704,513, filed Sep. 23, 2012, and Ser. No. 61/708,783, filed Oct. 2, 2012.

### BACKGROUND

### Prior Art

The middle ear contains a chain of small bones (ossicles) comprising the malleus, incus, and stapes. These bones transmit sonic vibrations from the eardrum to the inner ear where the vibrations are transformed into nerve impulses which then travel to the auditory center of the brain.

The ossicles are the smallest bones in the body and the only ones which are not surrounded by soft tissue. Instead, they live in an air-filled space. Their blood supply is limited by this circumstance, most particularly the middle bone (the incus), and this limitation imposes specific challenges for the surgeon who wants to attach a prosthesis to an ossicle to replace one which has become diseased. The mating of a prosthesis to an ossicle in a manner which will provide a secure, long-lasting attachment without loosening or damage to the ossicle has been a difficult issue in ear surgery for decades.

Prosthetic reconstruction of the ossicular chain is indicated in a variety of situations. For example, the disease otosclerosis (which causes abnormal formation of bone around the inner ear) commonly fuses the final bone in the chain (the stapes) to the bone surrounding the inner ear, resulting in hearing loss. The surgical correction of this condition requires a full or partial removal of the stapes and the attachment of a prosthetic stapes to one of the remaining ossicles to re-establish the connection between the middle ear and the inner ear. The success of this surgery is measured by the degree to which sound transmission from the prosthesis to the inner ear is restored in comparison to that which a healthy, perfectly functioning ossicular chain would provide.

In most cases, restoration of stapes function involves placement of the prosthesis around the final third of the long process (an arm-like extension) of the incus. The prosthesis comprises a loop or other attachment element connected to a vibration transmission element, which in turn is connected to a piston which moves the fluids of the inner ear to replicate the action of a normal stapes. A firm attachment of the prosthesis to the long process of the incus is critical. If the prosthesis is not firmly engaged to the long process, then the motion of the incus will not be adequately transmitted to the inner ear and the patient will not achieve a good hearing outcome. Moreover, any asynchronous motion resulting from a loose connection between the prosthesis and the incus will likely cause damage to the incus because of repetitive mechanical trauma. This repetitive trauma can result in erosion of the incus and further loosening of the prosthesis. A vicious cycle started in this manner can lead to eventual total failure of the prosthesis-incus attachment and a total conductive hearing loss.

Conversely, when the connection between the stapes prosthesis and the incus is too tight, failure can occur because delicate mucosa and blood vessels lying on the incus' surface can be compressed by the prosthesis, causing necrosis (tissue death) of the incus. Loss of bone tissue in the area of the prosthesis attachment can then cause loosening of the prosthesis, leading to eventual total prosthesis failure through the mechanism described above. These problems can be potentially mitigated by a design in which broad contact between the prosthesis and the ossicle to which it is mated is avoided via an attachment technology (trademarked OtoGrip) described in my Ser. No. 13/524,515 application.

Achieving a prosthesis-ossicle attachment which is sufficiently firm to translate ossicular motion without a significant loss of mechanical energy, yet loose enough to avoid any damage to the ossicle, is therefore a delicate balancing act. Innovations over the past several years to overcome this problem have included attachment loops which are comprised of softer, broader materials to distribute compressive forces over a wider area, as well as prostheses with limited contact patches against the ossicle. However, loosening and ossicular damage can still occur, in part because crimping (manual tightening of the prosthesis around the ossicle) is technically challenging. Surgeons may tend to undercrimp because of fear of ossicular necrosis, yet inadvertently increase the chances of necrosis because of a too-loose prosthesis. Undercrimping may also result in failure to achieve an optimum hearing outcome because of the mechanical energy losses associated with a loose prosthesis.

Manual crimping can be difficult for several reasons. These include the fact that the shape and diameter of the incus can vary significantly among individuals. Since the loop or other attachment element of various stapes prostheses has not yet been offered in variable sizes, this means that a loop of fixed size must be crimped to a variable degree in different individuals. Those with a large incus will require a smaller degree of absolute crimping compared to individuals with a small incus. There is also very little visual or tactile feedback for the surgeon in judging the optimum crimping pressure.

In some cases crimping can also change the prosthesis's final length, depending upon the orientation of the prosthesis and the final prosthesis shape after crimping. Unwanted shortening of the prosthesis can occur if that portion of the ossicular attachment loop contiguous with the vibration transmission element is pulled laterally and/or posteriorly by the crimping action. This can increase the chance that the piston will seat in the footplate fenestra (the hole created in the stapes footplate) in too shallow a fashion and later cause hearing loss or a fistula if the piston migrates out of the fenestra. Conversely, a crimp which changes the circular attachment loop into more of an oval shape in the lateral-medial dimension can lengthen the prosthesis such that the piston penetrates too far into the inner ear, which can result in vertigo.

Efforts to solve the problems associated with manual crimping have focused on the use of prostheses which do not require it. One such prosthesis is made of nitinol, a nickel-titanium alloy which can return to a predetermined shape upon heating. A nitinol loop with a closed gap is first carefully bent open by the surgeon and then placed loosely around the incus. Heat is then applied to the loop with a laser or other heat source, causing the loop to return to its closed position. However, problems have occurred with the loop “uncrimping” over time, leading to prosthesis loosening and hearing loss. Also, nitinol may have the potential to cause ossicular necrosis due to thermal injury to the ossicle after the loop has been heated.

Other prostheses which do not require manual crimping feature attachment elements comprising either a low elastic modulus material (superelastic nitinol) or a design which increases the deformability of the attachment element. These approaches allow the loop or other attachment element to be pushed onto the ossicle, thereby forcing the ossicle to deform it temporarily during the placement phase. One potential and significant complication associated with this placement technique is dislocation or other damage to the ossicle. This potential danger may result in a requirement for a larger attachment element opening than would otherwise be desired in order to diminish the force required to deform the prosthesis while pushing it on to the ossicle.

The ability to easily guide a prosthesis into position within the middle ear space has been another longstanding goal in the field of ear surgery. Thus far, attempts to accomplish this objective have had very limited success.

### SUMMARY

According to one aspect, I provide a middle ear prosthesis in which the ossicular attachment element is dimensioned and configured to at least partially surround an ossicle of the middle ear, with an opening at one side to receive an ossicle, and with a vibration transmission element connected to the ossicular attachment element. The prosthesis further comprises a plurality of recesses which are formed by, or on, the structures of the ossicular attachment element or vibration transmission element, either alone or in combination with one another. These recesses can accommodate the jaws of an instrument such as a forceps for securely engaging the prosthesis so that it can be easily guided into the middle ear during surgery. Engagement of the recesses also potentially allows the instrument to open (increase the diameter) and/or close (decrease the diameter) of the prosthesis' loop or other attachment element to facilitate placement of the prosthesis on to an ossicle.

### BRIEF DESCRIPTION OF THE DRAWINGS

FIG. 1 is a perspective view, not drawn to scale, of a typical prior-art loop stapes prosthesis.

FIG. 2 is a perspective view, not drawn to scale, of one potential embodiment of my ossicular prosthesis with recesses at the superior and inferior aspects of the of the ossicular engagement loop.

FIG. 3 is a perspective view, not drawn to scale, of a second potential embodiment of my ossicular prosthesis with recesses formed by angled projections arising from the ossicular attachment element.

FIG. 4 is perspective view, not drawn to scale, of a third potential embodiment of my ossicular prosthesis with an inferior recess formed by a projection arising out of the vibration transmission element.

FIG. 5 is a perspective view, not drawn to scale, of a fourth potential embodiment of my ossicular prosthesis in which the recesses are formed in a complex shape.

FIG. 6 is a perspective view, not drawn to scale, of a fifth potential embodiment of my ossicular prosthesis with an inferior recess formed by an angled transition between the ossicular attachment element and the vibration transmission element.

FIG. 7 is a perspective view, not drawn to scale, of a sixth potential embodiment of my ossicular prosthesis with an ossicular attachment element featuring an accordion-like variation in the attachment element shape as well as attachment projections arising from the ossicular attachment structures.

FIG. 8 is a perspective view, not drawn to scale, of the prosthesis embodiment from FIG. 4 with its ossicular attachment element an open position with recesses engaged by a forceps or other instrument.

FIG. 9 is a perspective view, not drawn to scale, of the prosthesis embodiment from FIG. 4 with its ossicular attachment element in a closed position with recesses engaged by a forceps or other instrument.

FIG. 10 is a perspective view, not drawn to scale, of a forceps which can engage and both open and close an ossicular attachment element for placement on to an ossicle.

FIG. 11 is a perspective view, not drawn to scale, of an engaging instrument with an outer sleeve and an inner sliding portion which can be used to engage a prosthesis for placement on an ossicle.

FIG. 12 is a perspective view, not drawn to scale, of an engaging instrument which demonstrates a variation of design in the sleeve portion.

### DETAILED DESCRIPTION

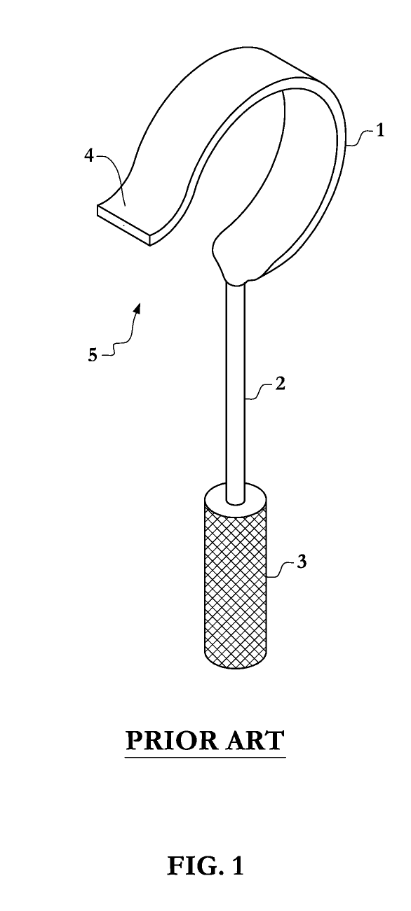

### FIG. 1

### Prior Art

FIG. 1 is an illustration of a typical loop stapes prosthesis which has been well described in the prior art. The loop and other attachment element designs described in this and all subsequent drawings can comprise either a round- or flat-wire configuration, otherwise known as a band or ribbon. The prosthesis has a loop 1 with a gap 5 for ossicular engagement. Loop 1 is contiguous with a vibration transmission element or arm 2 which terminates in a piston 3. Piston 3, or another component such as a flat “shoe,” can be placed either against the stapes footplate, on a piece of tissue covering the stapes footplate, or in a hole (fenestra) through the stapes footplate for translation of ossicular motion to the fluids of the inner ear. Alternatively, arm 2 in this and other designs shown can be attached to a second ossicular attachment element such that vibrations of the first ossicular attachment element can be transmitted to another ossicle. Many loop prostheses have a small “shepherd's crook” extension 4 at the terminus or free upper end of loop 1. This crook can form the superior border of gap 5 to facilitate a sliding placement of the attachment element over the ossicle, typically the incus.

The words “superior” and “inferior” or “upper” and “lower” are used as a semantic convenience to identify features of the devices illustrated that are closer to and further away from the top of the page, respectfully, and are not meant to denote the position of the prosthesis or other device components with respect to their orientation in the middle ear.

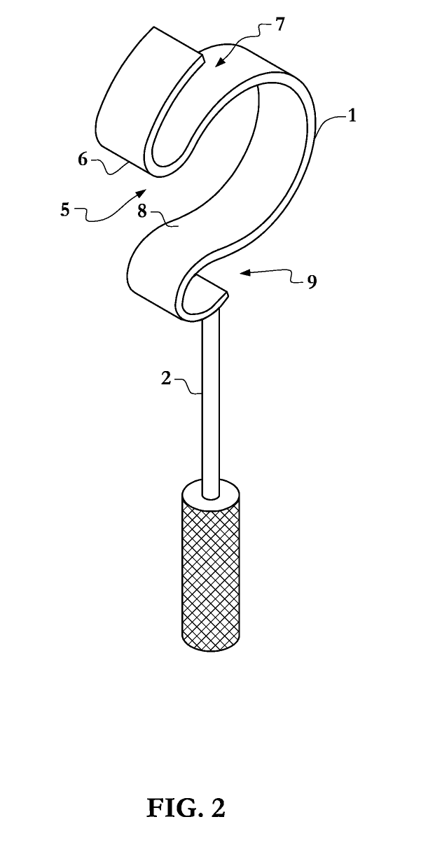

### FIG. 2

### Embodiment with Superior and Inferior Recesses Formed by Turns of Ossicular Attachment Loop

FIG. 2 illustrates one potential embodiment of my prosthesis. Loop 1 can be seen to make an additional turn upward and back at its upper terminal portion where crook 4 of FIG. 1 would normally continue down, thereby creating a bight 6 and a first and superior recess 7. Bight 6 is formed by a turn of the loop 1 at its terminus of greater than 90°; in the embodiment shown this turn is as shown approximately 180°. This illustrates one means of creating superior recess 7 which can accommodate the jaw of an instrument such as a forceps. When a jaw of the forceps is inserted into the recess it can securely engage the prosthesis and potentially provide a force to open loop 1. A more angular or squared-off bight 6 can be formed if desired by extending the superior terminus of loop 1 in such a way that it makes an abrupt angle or series of angles, rather than the gradual arc as illustrated.

Also, an inferior and second recess 9 is formed by an extension 8 of loop 1 past the long axis of arm 2, followed by an angled transition of extension 8 back to arm 2. A total angle of greater than 90° is formed (also shown as approximately 180°) between the extended inferior terminus 8 of loop 1 and arm 2.

Recesses 7 and 9 are open on three sides since they are not formed from closed loops. However these recesses and all other recesses can be “closed”, i.e., they can be formed from loops that are closed by extending the band further so that it meets the main loop (not shown). When a recess is open as shown in FIG. 2, the jaws of a forceps can be inserted into the respective recesses via the open part of the loop or from the side of the loop. However when a recess is closed on all but the two lateral sides, the jaws can be inserted into the loops from the side only. It would similarly be possible to close either or both of the sides of the recess; if both sides were closed, a blind pouch would be formed and the recess could only be engaged through the one open area between the sides.

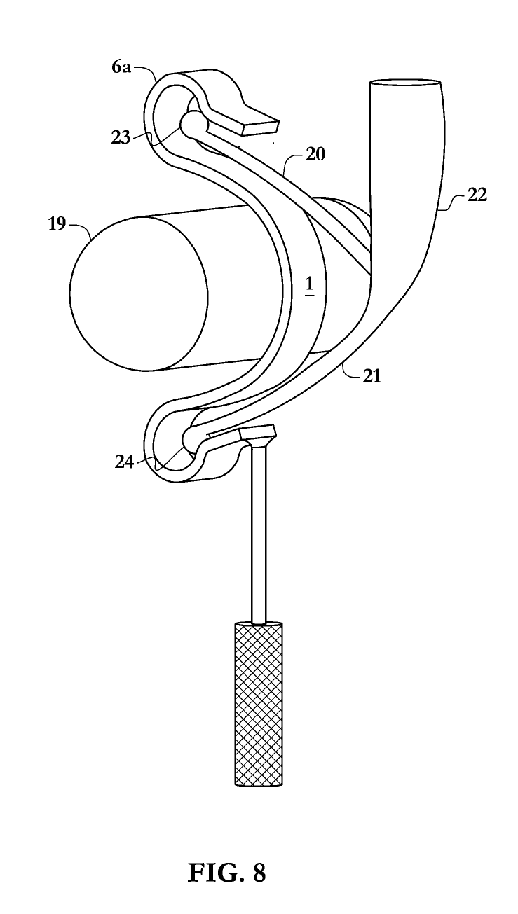

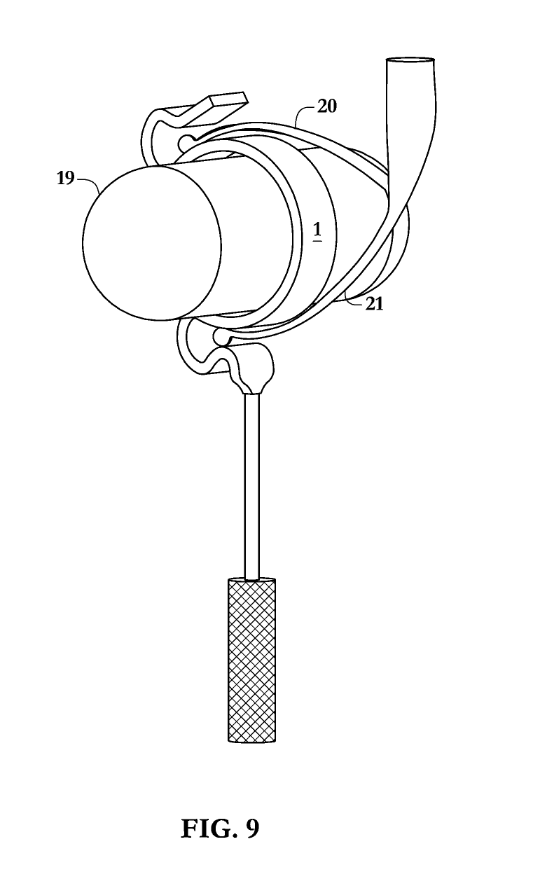

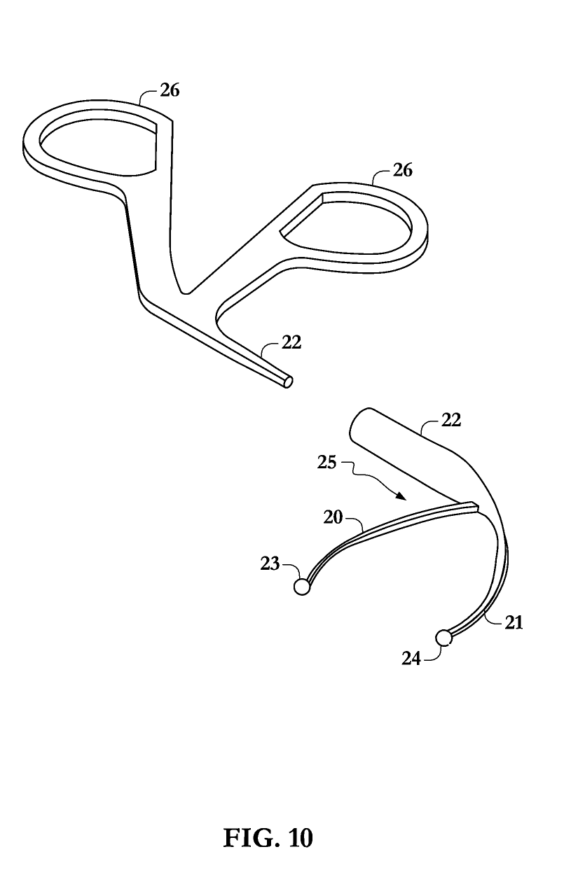

A forceps or other instrument having jaws can engage upper recess 7 and lower recess 9 to obtain a secure attachment to the prosthesis. (Suitable instruments are shown in FIGS. 10-12 and the operation of the forceps is shown in FIGS. 8 and 9). Moreover, this secure attachment can allow the instrument to spread apart/open loop 1 in such a manner as to enlarge opening 5 to fit the prosthesis onto an ossicle, or potentially provide a compressive force if needed.

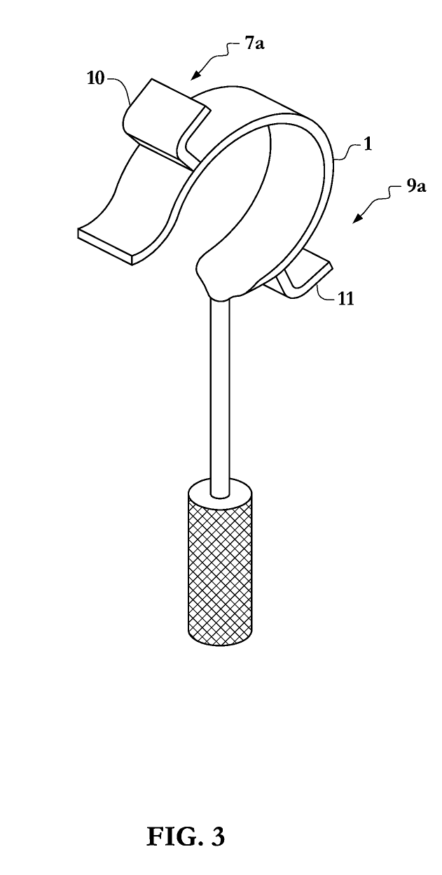

### FIG. 3

### Embodiment with Recesses Formed by Projections Arising from Ossicular Attachment Element

FIG. 3 illustrates another potential embodiment of my prosthesis. In this case an upper angled projection 10 and a lower angled projection 11 arise from and attach to loop 1 in such a manner that corresponding recesses 7a and 9a are formed. Projections 10 and 11 each comprise two sections joined at a right angle, with the end of one section proximal to the loop joined at a right angle to the outside of the loop so that the section distal from the loop is roughly parallel to the loop. It would of course be possible to round the angle of projections 10 and 11 such that they form a smooth arc rather than the angular appearance as shown.

In any case, there are a large variety of ways to alter the structure of the ossicular attachment element or the vibration transmission element to create embodiments with recesses suitable for engagement of the prosthesis by a forceps or other instrument.

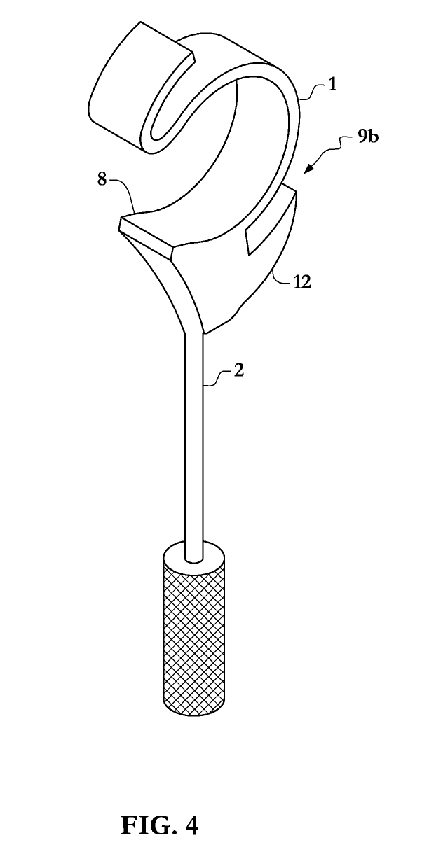

### FIG. 4

### Embodiment with Inferior Recess Formed by Projection from the Vibration Transmission Element

FIG. 4 shows another embodiment where the superior portion of the prosthesis loop is identical to that shown in FIG. 2, but a lower recess or slot 9b is formed by the inferior attachment of loop 1 to arm 2 and by a projection 12 arising from arm 2. This design for a lower recess may offer some advantages over that shown in FIG. 2 if that embodiment allows some mechanical energy losses through flex of the structure comprising the lower recess. It can be appreciated that the embodiment in FIG. 4 would be entirely rigid as the motion of loop 1 would be transmitted entirely to arm 2 and not allow any such flex.

Recess 9b need not be straight and need not be a simple curve. If desired, it can assume a complex shape and thereby precisely fit the jaw of an engaging instrument which has a shape to form-fit the recess. As in FIG. 2, extension 8 of loop 1 provides greater contact with that surface of the ossicle nearest to the vibration transmission element.

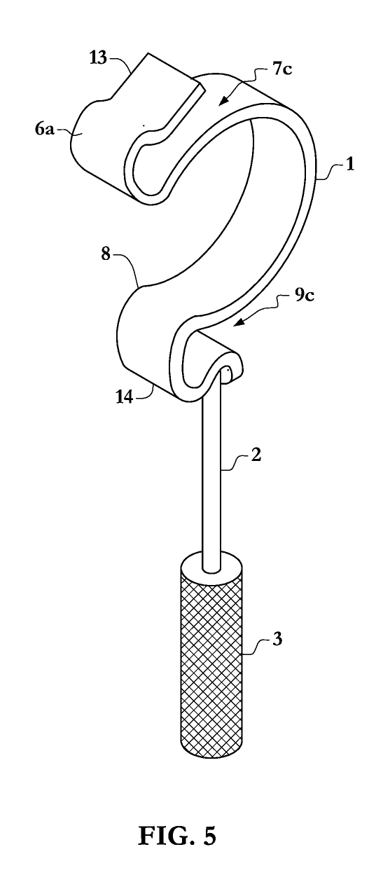

### FIG. 5

### Embodiment with Recesses Manifesting a Complex Shape

FIG. 5 shows an embodiment with different superior and inferior recesses. In this case superior bight 6a in comparison to bight 6 of FIG. 2 may form a complex shape, whereby the loop circumscribes an arc as a termination of loop 1 of greater than 180°. In the example shown it is approximately 260°. The resultant recess 7c may or may not be extended by a continuation 13 of loop 6a. Recess 7c in comparison with recess 7 of FIG. 2 has an additional superior space as shown which may accommodate a projection from the superior jaw of a forceps or similar instrument. This design enhances the secure engagement of a jaw which incorporates a protuberance on its upper surface such that the protuberance can fit securely in recess 7c. When a forceps or other engaging instrument is engaged to form-fit superior loop 6a in this manner, any distractive opening force exerted on loop 1 can be executed in a more secure fashion than otherwise with less of a chance for the jaw to slip out of recess 7c when engaged with loop 6a.

The inferior recess 9c has an additional inferior extension of space defined by a bight 14 which describes an arc of greater than 180° from extension 8 of loop 1. Recess 9c can thereby accommodate a protuberance from the lower jaw of an engaging instrument in a similar manner as recess 7c. The transition from inferior loop 14 to arm 2 follows a curve in this illustration but can be sharply angled if desired. In either case, arm 2 and piston 3 (or a second ossicular attachment element if the prosthesis attaches to a second ossicle) are optimally placed directly under the central area of loop 1. Thus when an ossicle is engaged by the loop, the vector of ossicular movements corresponds to the long axis of the vibration transmission element (arm) and piston or second attachment element.

The shapes of loops 6a and 14 and their corresponding upper and lower recesses 7c and 9c need not be as shown but rather can have numerous different designs, such that the contours of the jaw of a forceps or other engaging instrument can form-fit the complex shape of the recesses and thereby achieve a more secure attachment to the prosthesis. For example, the edges of the ribbon at the upper and lower recesses can be bent to create an inward curvature of the ribbon, such that more of a closed-pocket configuration can be achieved for recesses 7c and 9c. Alternatively, a recess with a fully closed pocket can be achieved by including a wall bridging both sides of the upper and lower recesses. This configuration can potentially offer a more secure connection between the prosthesis and an instrument engaged in the recesses, since a lateral slipping movement of the instrument out of the recess would be blocked by the lateral walls. Joining the upper and lower aspects of the inferior loop by the integration of lateral walls would have the additional advantage of eliminating any potential spring effect created by flexion of lower loop 14 when loop 1 moves up and down with the ossicle to which it is attached. Any such spring effect would lead to some mechanical energy loss between the movement of loop 1 and arm 2.

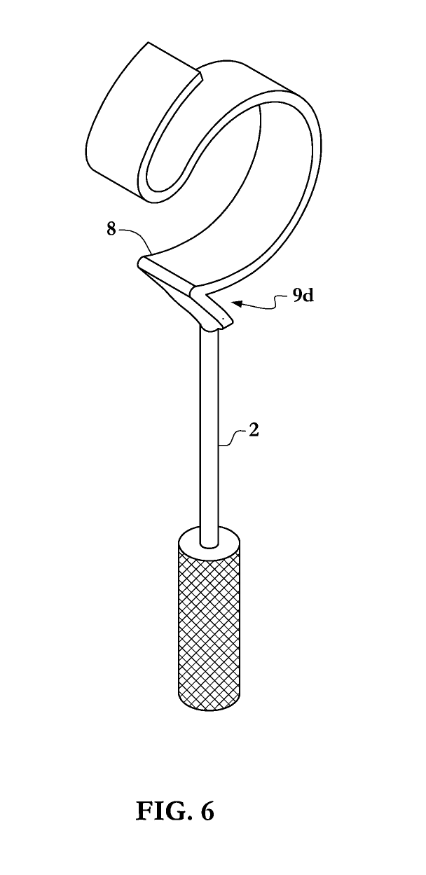

### FIG. 6

### Embodiment with an Angled Variation of the Lower Recess

FIG. 6 shows a prosthesis according to another embodiment of the design. A recess 9d is formed by an acutely angled transition of loop extension 8 to arm 2. The shallow, triangular recess thus formed may be more rigid than other embodiments but will not allow the same degree of purchase required if a distracting force is applied by the inferior jaw of an instrument engaged in the recess. This of course can be rectified by the placement of a projection from arm 2, such as projection 12 in FIG. 4.

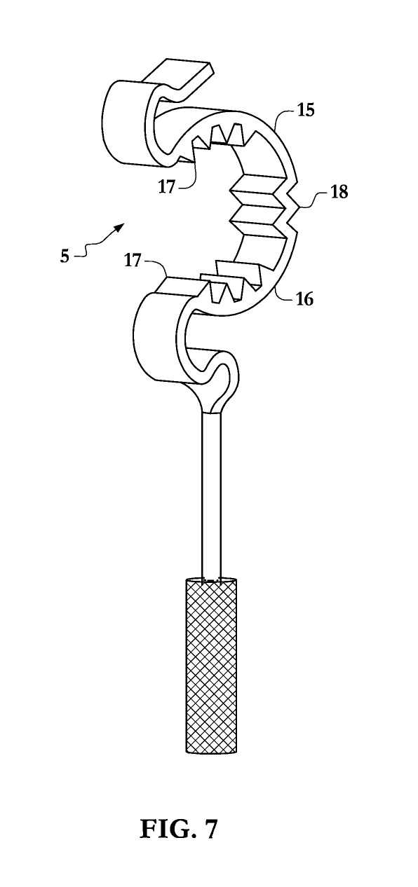

### FIG. 7

### Alternative Prosthesis Attachment Element Design

FIG. 7 shows an ossicular prosthesis illustrating another embodiment of the ossicular attachment element. The upper and lower recesses of this prosthesis are similar to those illustrated in FIG. 5, but loop 1 has been replaced by upper and lower clamshell-shaped structures 15 and 16 which can serve as mating surfaces with an ossicle. Also illustrated are optional projections or spikes 17 (see my above '515 co-pending application) which line the upper and lower inside margins of structures 15 and 16. These are designed to limit the contact area the attachment element makes with the ossicle to which it is mated. The projections are shown to demonstrate compatibility of the prosthesis design with the technology described in my above '515 application. If the projections are not attached as shown, then structures 15 and 16 serve as a direct means of contact with the ossicle to which the prosthesis is mated. Opposite attachment element opening 5 is an accordion- or zig-zag-shaped portion 18 which joins ossicular mating surfaces 15 and 16 together. Structure 18 will reduce the force necessary to change the size of opening 5 during the procedure for the placement of the prosthesis on to an ossicle. The number and size of the folds of structure 18 may be changed as needed to adjust both the opening pressure desired as well as final closing pressure the attachment element would exert on an ossicle. The inclusion of structure 18 expands the choice of materials with higher elastic moduli which can be used to fabricate the prosthesis, since a prosthesis of this design can more readily return to its shape after opening if compared to a design without this feature.

### FIGS. 8 and 9

### Placement of Prosthesis on an Ossicle

FIG. 8 is an illustration of an ossicular prosthesis loop attachment element of the design shown in FIG. 5 with its upper and lower recesses being engaged by the jaws of a forceps or other suitable applicator instrument. In this and the following illustration, the jaws of the applicator and the inside walls of the prosthesis' recesses would be in physical contact with one another but this is not shown for the sake of illustrative clarity. Also, the handles or hand piece of the applicator are not shown.

An ossicle 19 is engaged by prosthesis loop 1. In the case that the prosthesis is designed to “self-crimp” via its elastic modulus, it is urged into an open position by moveable jaws 20 and 21 of the instrument. A shaft 22 joins the jaws to the hand piece for control by the surgeon. A protuberance 23 at the terminal portion of upper jaw 20 of the instrument can fit securely in the recess formed by loop 6a. Protuberance 23, once engaged in superior recess created by loop 6a, can help to prevent upper jaw 20 from disengaging from the prosthesis when it is in the opened position. Lower jaw 21, with its protuberance 24, can securely engage the lower recess in the same manner as the upper recess is engaged by the upper jaw 20. The forceps' jaws as illustrated are more rounded in shape but they can have a more flattened ribbon shape to match the shape of the prosthesis' recesses to further increase the security of the jaw-recess interface. The applicator, once engaged with the prosthesis, is in a position to exert both expanding and compressive forces on the attachment element.

Once the applicator engages the prosthesis, a very secure attachment can be achieved so that the prosthesis will be “loaded” on to the instrument, as is known to those familiar in the art. This loading confers additional advantages in terms of ease of use and accuracy in the placement of the prosthesis on the ossicle. In furtherance this objective, I anticipate that the maximal opening of the engaging instrument's jaws can be limited by the instrument's design so that maximal movement of the instrument handle in the open position will open the prosthesis attachment element only enough to achieve placement on to the ossicle and no more. In this way the surgeon's hand can exert a maximal opening force to the instrument without having any concern that the prosthesis may be forced open in an excessive manner, for example to such an extent that the instrument's jaws may slip off the prosthesis or, in the case of a self-crimping prosthesis, that the modulus of the attachment element would be exceeded such that the prosthesis would fail to return to its native, unopened configuration when the jaws of the instrument were closed. If the prosthesis is offered in a manner such that it is already joined to an applicator in the surgical package, then it may be most advantageous for the applicator and prosthesis to be set in the maximally opened position. The surgeon could then simply close the applicator and prosthesis's attachment element once the prosthesis is guided into position on the ossicle.

FIG. 9 is an illustration of an ossicular prosthesis loop attachment element of the design shown in FIG. 8, demonstrating how loop 1 assumes a closed configuration once it has been applied to ossicle 19. As illustrated, upper and lower jaws 20 and 21 are closed in comparison to the open state in FIG. 8. In this position the instrument can either release from the attachment element or provide an expanding force to open the attachment element so that the attachment element can then be removed from the ossicle. The design allows significant advantages once the prosthesis has been placed on the ossicle as shown. Specifically, if after placement the prosthesis is seen to be in a suboptimal position on the ossicle, then the instrument's jaws can re-engage the prosthesis and open the attachment element, allowing easy repositioning or removal of the prosthesis. A prosthesis may need to be removed and replaced if it is discovered to be too long, too short, not accurately placed in the fenestra of the footplate, or if complications occur after surgery. It can be difficult and time consuming to remove, reposition, or replace a conventional or heat-activated nitinol prosthesis once it has been crimped, and damage to the ossicle can occur during this process as well.

### FIG. 10

### Forceps with Jaw Variation to Engage Prosthesis

FIG. 10 illustrates a complete view of an engaging instrument, in this case a forceps, which is shown in a partial view in FIGS. 8 and 9 and which demonstrates the engaging jaws which can form-fit a prosthesis. The basic forceps design is well-known to those familiar and skilled in the art. The forceps has two handles with respective finger holes 26. The handles are hinged together (hinge not shown) and are connected to a conventional motion-transmitting lever (not shown) which moves along a shaft 22 in response to opening and closing of the handles. The part of shaft 22 distal from handle and finger holes 26 is enlarged in the illustration. Motion of the finger holes 26 in relation to one another moves the lever axially along shaft 22, which in turn moves jaws 20 and 21 closer together or further apart. Jaws 20 and 21 can form a curve to fit to the rounded aspects of loop 1 or other attachment element. Upper jaw 20 has a protuberance 23 which can help assure a secure fit with superior recess 7c seen in prior illustrations which is designed to accommodate it. Similarly, lower forceps jaw 21 has a protuberance 24, which can form fit with lower recess 9c. The jaws of the forceps or other engaging instrument can also have other shapes in order to form-fit a prosthesis' recesses which are designed to accommodate them.

Angle 25, which jaws 20 and 21 form with shaft 22, can vary according to the orientation of the prosthesis' ossicular attachment element which the instrument is designed to engage. For example, if the ossicular attachment element of a stapes prosthesis is designed to engage an incus in more of a posterior-anterior direction, with an opening facing more anteriorly than in a lateral-medial direction, then angle 25 will be correspondingly greater. A more posterior-anterior oriented ossicular attachment element which is engaged by an appropriately angled forceps is illustrated in FIGS. 8 and 9. It is also possible to design the jaws of the forceps or other engaging instrument to face backwards if the surgeon wants to attach the prosthesis in such a way that the ossicular attachment element opening faces in a posterior direction. This could be desirable in some situations in which the space in the posterior middle ear is very tight due to an individual anatomic variation.

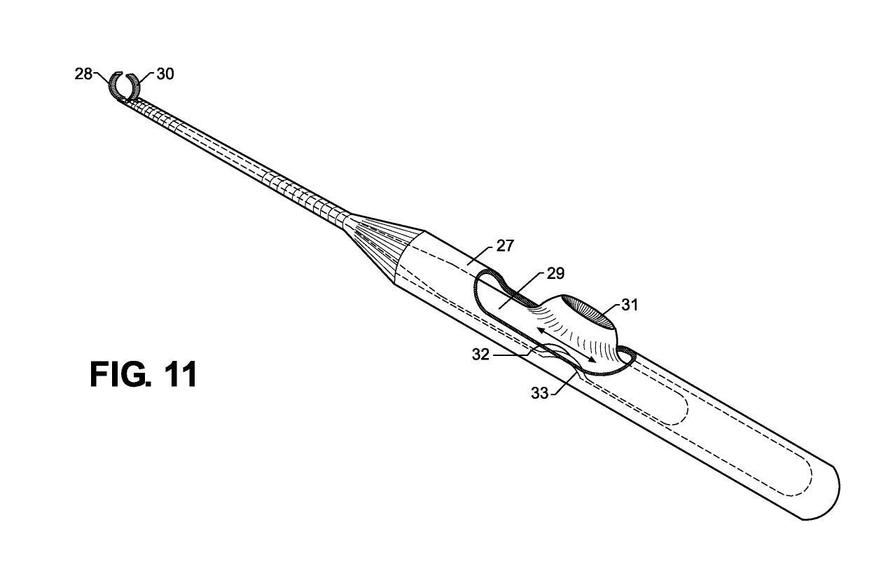

### FIG. 11

### Alternative Applicator Design

FIG. 11 illustrates an alternative engaging tool or applicator which comprises a fixed sleeve containing a movable sliding component within the sleeve. Sleeve 27 terminates in a fixed distal jaw 28 which is designed to engage the lower recess of an ossicular prosthesis. A movable sliding arm 29 within the sleeve terminates in an upper jaw 30 which moves with the rest of the sliding component. Movement of the sliding component can be precisely controlled via longitudinal pressure on a finger interface 31 of the sliding component. Arm 29 can be designed to be held in a position through placement of an interlocking tab 32 on a protuberance 33 built in to the sleeve as shown, until pushed off this position by finger pressure. This will allow a prosthesis and applicator to be mated to one another and then packaged in a semi-locked opened position. The surgeon can then use the applicator to guide the prosthesis into position on an ossicle, close the prosthesis' attachment element through motion of the applicator at the finger interface 31, and thereby attach the prosthesis to the ossicle

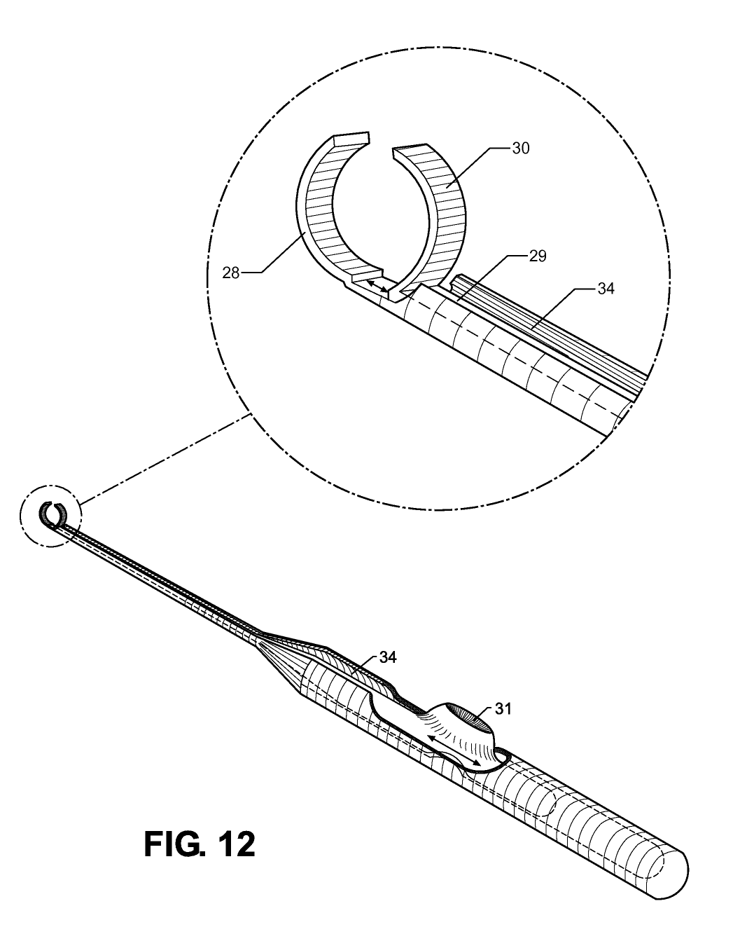

### FIG. 12

### Alternative Applicator Design with Open Sleeve

FIG. 12 illustrates another embodiment of the prosthesis applicator. In this case a sleeve 34 is characterized by an open configuration from the area of the slider's finger rest 31 to the terminal portion of the instrument where jaws 28 and 30 are located. Sliding element 29 terminates in an upper jaw 30 which is freely movable while jaw 28 remains fixed. If the sleeve is in an open configuration as shown, this will allow an easier assembly of the device since the sleeve and the sliding component can be snapped together after separate fabrication, which may reduce costs.

The applicators in FIGS. 11 and 12 can be manufactured relatively inexpensively from plastic or similar material and be disposable. Disposability can further simplify a surgical procedure involving the placement of a prosthesis of the type described and reduce costs.

### CONCLUSIONS, RAMIFICATIONS, SCOPE

The prosthesis designs and the prosthesis engagement instruments described address the problems presented previously via an integrated approach. First, for self-crimping prostheses, the issue of varying ossicular size and diameter among different individuals can be managed by the use of prostheses with loops or other attachment element designs of differing inner diameters. This will allow the surgeon to provide a “custom” fit prosthesis such that ossicles with varying diameters will be matched and mated to prostheses with specific diameters to provide optimum pressure on the ossicle. Optimum pressure is that which is low enough to minimize the likelihood of ossicular damage such as pressure necrosis while being firm enough to transmit as much ossicular motion as possible to the prosthesis for the best hearing result.

The prosthesis here incorporates spaces (recesses) formed by the structure of the ossicular attachment element alone or in combination with the vibration transmission element such that the recesses can be engaged by the jaws of a forceps or other instrument, otherwise referred to as an applicator. In addition to a forceps, which has hinged handles, the applicator can be hingeless, as is the sliding-arm applicators of FIGS. 11 and 12.

Engagement of the prosthesis via its recesses can simplify the handling of the prosthesis and may eliminate the requirement for manual crimping if the loop or other prosthesis attachment element is comprised of a material with a suitable elastic modulus. In this circumstance, placement of the prosthesis can be accomplished by initially “uncrimping” or opening the loop or attachment element via a distractive force applied by the engaging instrument. In the opened or “uncrimped” position, the prosthesis' attachment element can be guided into place around the ossicle. The engaging instrument can then be gently closed, allowing the elastic modulus of the prosthesis material to return the attachment element to its original size and shape and engage the ossicle in a predictable, secure manner with a predictable pressure. An engaging instrument can provide a compressive crimping force if needed as well.

Utilization of the elastic modulus of the prosthesis' loop or other attachment element for “self” crimping can simplify the mating of the prosthesis to the ossicle. The problems associated with traditional manual crimping described earlier could be entirely avoided with this method. The surgeon would be relieved of the difficult decision of when a prosthesis has been crimped to the optimal pressure and would no longer need to worry about changes in the prosthesis' overall length or shape as the result of crimping; the prosthesis would look the same after placement in the patient's middle ear as it did coming out of the box. Moreover, since the prosthesis would crimp itself, the disadvantages of using heat-activated nitinol which requires a laser or other form of heat activation for loop closure would be avoided, saving operating time, cost, and possible complications due to inadvertent application of the laser or other heating element to nearby structures. Lastly, a design in which the elastic modulus of the attachment material results in self-crimping is also entirely compatible with the attachment technology described in my above '515 application.

The instrument's jaws and the prosthesis' recesses can be specifically designed to fit together and allow precise engagement of the prosthesis by the instrument. This design can allow both opening and closure of the attachment element with motion of the fingers at the handle of the instrument engaged with the prosthesis. An additional benefit is that a secure grasp or “loading” of the prosthesis by the jaws of the engaging instrument is provided to improve the ease of handling and placement in the middle ear, thus reducing operating time.

The ossicular prosthesis can either be loaded on to an engaging instrument/applicator at the time of surgery, or alternatively, packaged in a pre-loaded fashion whereby it would be already joined with the instrument. The applicator can be fabricated from an inexpensive material such as plastic and be designed as disposable. In a pre-loaded configuration, the surgeon will be relieved of the step of applying the applicator to the prosthesis, which will save operating time. If the applicator were to engage the prosthesis in the opened, distracted position out of the box, the surgeon would be able to simply remove the applicator and prosthesis as a single unit from the packaging, guide the prosthesis into position on to the ossicle, close the applicator, and then withdraw the applicator from the prosthesis and the surgical field thereby completing this part of the procedure in an expeditious manner.

While particular embodiments have been described, it is intended that the embodiments should not be limited thereto. Those skilled in the art will recognize that many variations are possible. For example, although the figures illustrating prostheses show a loop or clamshell-shaped ossicular attachment design and recesses which are open on three sides, other prosthesis shapes and designs may employ the principles described. The prostheses described may be thought of comprehensively as the entire prosthesis or alternatively, as the ossicular attachment element and/or the vibration transmission element of a prosthesis which are dimensioned and configured to form a plurality of recesses which can be engaged by an instrument.

Other embodiments for prostheses utilizing the novel design described can be utilized in situations other than those involving attachment of a stapes prosthesis to the incus, or of a prosthesis joining the malleus to the stapes or the stapes footplate. Thus other situations requiring secure attachment of a prosthesis to an ossicle can utilize the designs disclosed. In the same manner, instruments other than the distracting instruments represented here can employ the principles to achieve engagement with a prosthesis.

Thus the scope should be determined by the appended claims and their legal equivalents, and not by the particulars shown or described.

1. A middle ear prosthesis, comprising:
an ossicular attachment element having an opening dimensioned and configured to at least partially surround and embrace an ossicle of a middle ear,
a vibration transmission element connected to the ossicular attachment element, and
a plurality of recesses formed on or by the structure of the ossicular attachment element or the vibration transmission element, or both,
the recesses having a predetermined shape and design for operably mating with the jaws of a predetermined forceps or other applicator instrument so that the applicator instrument can be used to securely engage the prosthesis via the recesses for controlled guidance and attachment of the prosthesis to the ossicle.

2. The middle ear prosthesis of claim 1 wherein the predetermined forceps or other applicator instrument can be used to vary the size of the ossicular attachment element after engagement of the instrument with the recesses of the prosthesis.

3. The middle ear prosthesis of claim 1 wherein the vibration transmission element terminates in a piston or other structure for placement in, or adjacent to, the fluids of the inner ear so as to pass vibrations from the ossicular attachment element to the fluids of the inner ear.

4. The middle ear prosthesis of claim 1 wherein the ossicular attachment element has a terminal portion not contiguous with the vibration transmission element and at least one of the recesses is formed by an extension of the terminal portion, the extension making an angle of greater than 90 degrees with the terminal portion.

5. The middle ear prosthesis of claim 1 wherein the ossicular attachment element has an extended terminal portion that is contiguous with the vibration transmission element, and at least one of the recesses is formed by an extension of the terminal portion, the extension having a turn such that an angle of greater than 90 degrees is formed between the extension and the attachment of the extension to the vibration transmission element.

6. The middle ear prosthesis of claim 1 wherein one or more recesses are formed by a projection attached to, and extending from, the ossicular attachment element.

7. The middle ear prosthesis of claim 1 wherein one or more recesses are formed by a projection attached to, and extending from, the vibration transmission element.

8. The middle ear prosthesis of claim 1 wherein the one or more recesses are open on three sides.

9. The middle ear prosthesis of claim 1 wherein one or more recesses are open on two sides.

10. The middle ear prosthesis of claim 1 wherein one or more recesses are configured such that all their sides but one are closed, and the recess or recesses accordingly form a blind pouch.

11. The middle ear prosthesis of claim 1 wherein the ossicular attachment element is comprised of upper and lower surfaces of a concave clamshell shape having an opening for admittance of an ossicle into the attachment element, the surfaces being joined opposite the opening by an accordion-shaped structure which can flex open and closed with an expanding or compressive force applied to the attachment element.

12. A method of attaching a prosthesis to an ossicle of the middle ear, comprising:
a. providing the prosthesis comprising recesses which can be engaged by an applicator instrument according to claim 1.
b. providing an applicator instrument which is dimensioned and configured to engage the prosthesis via the recesses of the prosthesis.
c. attaching the applicator instrument to the prosthesis so that the jaws of the instrument are engaged in an operable position to apply an expanding force directed away from the geometric center of the prosthesis' attachment element, or a compressive force directed toward the geometric center of the attachment element, or both;
d. guiding the instrument while engaged with the prosthesis into a position so the attachment element of the prosthesis partially surrounds an ossicle of the middle ear;
e. terminating the expanding force or applying a compressive force by the applicator instrument with resultant lessening of the diameter of the ossicular attachment element, and;
f. disengaging the applicator instrument from the prosthesis after the ossicular attachment element of the prosthesis is attached to the ossicle.

13. The applicator instrument of claim 1 wherein the instrument is comprised of a handle with finger holes or other interface for a surgeon's hand, a shaft attached to the handle, and jaws at the end of the shaft which are specifically configured, dimensioned and angled to engage the recesses of the prosthesis.

14. The applicator instrument of claim 13 wherein the upper jaw of the instrument is connected to a sliding element within a fixed sleeve, the sliding element arranged to be moveable in response to motion of a surgeon's finger or hand.

15. The applicator instrument of claim 14, further including a sleeve that fully encloses said sliding element.

16. The applicator instrument of claim 14, further including a sleeve that partially encloses the sliding element.
